(19) United States
(12) Patent Application Publication
(10) Pub. No.: US 2022/0354541 A1
(43) Pub. Date: Nov. 10, 2022

(54) SKELETAL SUPPORT MEMBER

(71) Applicant: Johannes Hendrik Davis, Stellenbosch (ZA)

(72) Inventor: Johannes Hendrik Davis, Stellenbosch (ZA)

(21) Appl. No.: 17/764,966

(22) PCT Filed: Sep. 30, 2020
(86) PCT No.: PCT/ZA2020/050054

§ 371 Date: Mar. 29, 2022

(30) Foreign Application Priority Data
Sep. 30, 2019 (ZA) 2019/06423

(51) Int. Cl. A61B 17/70

(52) U.S. Cl. CPC A61B 17/702; A61B 2017/00681

(57) ABSTRACT

This invention relates to a skeletal support member (1) and more specifically, but not exclusively, to a skeletal support member (1) for use in spinal or intramedullary surgery. In accordance with the invention there is provided a skeletal support member (1) comprising an elongate body with integrally formed inner (2) and outer (3) portions, at least one mechanical attribute of the inner portion (2) differing from a corresponding mechanical attribute of the outer portion (3), and the inner portion (2) being shaped and sized such that a corresponding mechanical attribute of the body varies longitudinally. It is envisaged that the invention will provide a skeletal support member (1) which has varying mechanical properties along the length thereof in at least one direction which may be specifically tailored to allow specific movement and apply corrective forces and moments which are bespoke to a patient.

### FIELD OF THE INVENTION

This invention relates to a skeletal support member and more specifically, but not exclusively, to a skeletal support member for use in spinal or intramedullary surgery.

### BACKGROUND TO THE INVENTION

Spinal fusion surgery, particularly when performed for the treatment of scoliosis or other forms of spinal deformation, make use of so-called spinal rods which are anchored to a patient's vertebrae and secured along the length of the patient's spine. The spinal rods are elastically deformed to conform the shape of the spine and to apply corrective forces and moments along the length of the patient's spine. This creates a rigid spinal structure and allows for limited post-surgery movement of the spine.

This problem has been addressed in the prior art a number of ways, some of which are discussed in more detail below.

U.S. Pat. No. 7,875,059 in the name of Warsaw Orthopedic, Inc. entitled “Variable Stiffness Support Members” discloses a support member made of interlocking sections of different materials to obtain a variable stiffness across the length of the composite rod.

A 2016 article by Vladimir Brailovski and others in the Journal of Shape Memory and Superelasticity entitled “Ti—Ni Rods with Variable Stiffness for Spine Stabilization: Manufacture and Biomechanical Evaluation” discloses a rod of titanium-nickel shape memory alloy which is annealed at varying temperatures along the length to provide variable flexural stiffness across the length of the rod.

European patent number 2 224 866 in the name of Zimmer Spine, Inc. entitled “Flexible Member with Variable Flexibility For Providing Dynamic Stability to a Spine” discloses a member with parallel grooves machined therein which is installed at various positions along the length of a conventional rod to alter the stiffness thereof at the installation positions.

### OBJECT OF THE INVENTION

It is accordingly an object of this invention to provide a skeletal support member which, at least partially, alleviates the problems associated with the prior art or provides a useful alternative thereto.

### SUMMARY OF THE INVENTION

In accordance with the invention there is provided a skeletal support member comprising:
an elongate body with integrally formed inner and outer portions;
at least one mechanical attribute of the inner portion differing from a corresponding mechanical attribute of the outer portion; and
the inner portion being shaped and sized such that a corresponding mechanical attribute of the body varies longitudinally.

The mechanical attributes include stiffness, density, strength, ductility, hardness, and/or surface finish.

The cross-sectional circumferential profile of the outer section is longitudinally consistent.

The inner portion may be manufactured from a different material or have a different structure to provide the differing mechanical attributes.

The structure of the inner portion may be a lattice, grid, or trabecular framework. The inner portion may be sintered. The inner portion may include struts and beams.

The attributes of the body at a longitudinal location is related to a ratio of the cross sectional area of the inner portion to the cross-sectional area of the outer portion at the position.

The ratio may be varied along the length of the member. The ratio may be lowest at a central part of the body and highest at the ends, or varied along the length to impart specific deforming or deformity opposing forces.

The inner portion may include multiple sections with distinct shapes combined along the length of the member to form the inner portion.

The inner portion may be formed by combining two opposing pyramidal, conical, frustopyramidal, or frustoconical sections with bases oriented towards the ends of the body and apexes oriented toward the centre of the body.

The outer portion may extend to the ends of the body and enclose the inner portion.

The outer portion may have a relatively lower surface roughness than the inner portion.

The longitudinally varying attribute may be represented as a profile of the attribute in a direction perpendicular to a longitudinal axis of the body along the length of the body.

The member may have different profiles in at least two directions perpendicular to the longitudinal axis of the body.

The member may be made of titanium and be manufactured using an additive manufacturing process such as laser metal deposition.

The body may be cylindrical with rounded ends and a solid outer portion.

The member may be 3D printed such that the outer portion transitions to the inner portion gradually or distinctly.

The inner portion may have relatively lower density and stiffness than the outer portion.

The body may have a relatively higher stiffness at the centre and relatively lower stiffness at the ends.

The member may be a spinal rod or an intramedullary nail or prosthetic stem.

### BRIEF DESCRIPTION OF THE DRAWINGS

An embodiment of the invention is described below, by way of a non-limiting example only, and with reference to the accompanying drawing in which:

FIG. 1 is a schematic side view, showing hidden detail, with sectional views E-E and F-F of a first embodiment of a skeletal support member;

FIG. 2 is a schematic side view, showing hidden detail, with sectional views E-E and F-F of a second embodiment of a skeletal support member;

FIG. 3 is a schematic side view, showing hidden detail, with sectional views E-E and F-F of a third embodiment of a skeletal support member;

FIG. 4 is a schematic side view, showing hidden detail, with sectional views E-E and F-F of a fourth embodiment of a skeletal support member;

FIG. 5 is a schematic side view, showing hidden detail, with sectional views E-E and F-F of a fifth embodiment of a skeletal support member;

FIG. 6 is a schematic side view with sectional view A-A and a sectioned perspective view of a sixth embodiment of a skeletal support member;

FIG. 7 is a schematic side view with sectional view A-A of a seventh embodiment of a skeletal support member; and

FIG. 8 is a schematic side view with sectional view A-A of an eighth embodiment of a skeletal support member.

### DETAILED DESCRIPTION OF THE DRAWINGS

With reference to the drawings, in which like features are indicated by like numerals, skeletal support member is generally indicated by reference numeral 1.

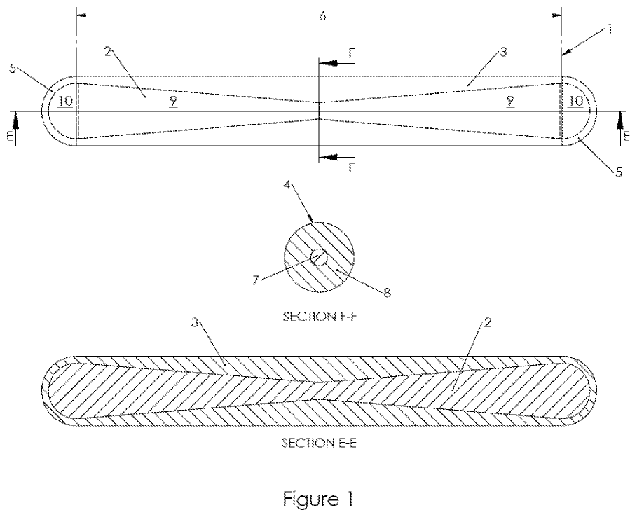

Eight embodiments of a skeletal support member 1 are shown in the accompanying drawings, one in each of FIGS. 1 to 8. Each embodiment includes an elongate body with integrally formed inner 2 and outer 3 portions. The body has a central part 6 with ends 5. At least one mechanical attribute of the inner portion 2 differs from a corresponding mechanical attribute of the outer portion 3. The mechanical attribute may be, for example, stiffness, density, strength, ductility, hardness, and/or surface finish. For the purposes of explanation herein reference will be made to the stiffness of the member. However, those skilled in the art will recognise that different mechanical properties are often associated with each other or may be varied to achieve a desired purpose. The inner portion 2 is shaped and sized such that the stiffness of the body varies longitudinally.

The embodiments shown in FIGS. 1 to 8 all have an outer portion with a cross-sectional circumferential profile 4 which is longitudinally consistent along the central part 6. Specifically, the cross-sectional circumferential profile 4 of the outer section 3 is, save for the ends 5, circular with a constant diameter and may be semi-circular or elliptical. The embodiments described herein are primarily aimed at use as spinal rods which commonly have circular cross-sectional profiles 4. Where the skeletal support member 1 is used for other applications, for example as a stem implant or a hip prosthesis, the cross-sectional profile 4 will vary considerably to accommodate the internal anatomical shape of the bone that it is implanted into.

The inner portion 2 may either be manufactured from a secondary material, wherein the mechanical properties of the secondary material will provide the variance in mechanical properties of the member 1 as a whole. Alternatively, the inner portion may be manufactured from the same material as the outer portion 3 and the internal structure of the inner portion provides the variance. Specifically, the inner portion may have an internal structure which includes a lattice, grid, or trabecular framework which forms the internal structure. The inner portion 2 may be sintered to create a granular internal structure or have struts and beams to provide variance.

Where the inner 2 and outer 3 portions are made of the same material, it is desirable that the member 1 be produced through an additive manufacturing process (commonly referred to as 3D printing) such as, in the case of commonly used titanium implants, laser metal deposition. The additive manufacturing process allows complex internal structures to be produced for the inner section 2 and may allow the outer portion 3 to transition to the inner portion 2 either gradually or distinctly.

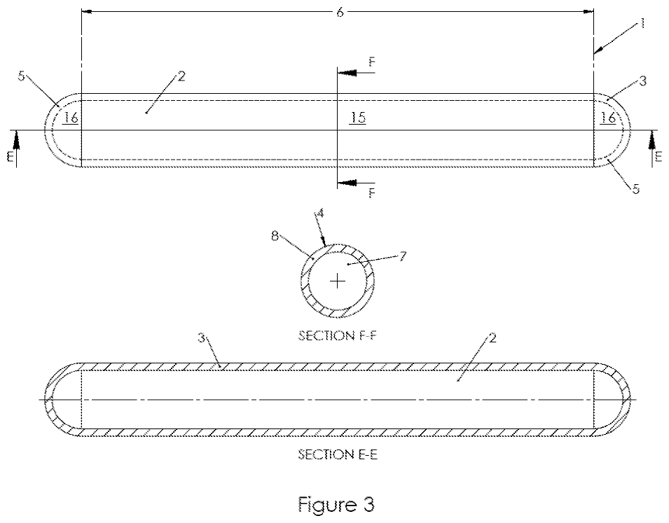

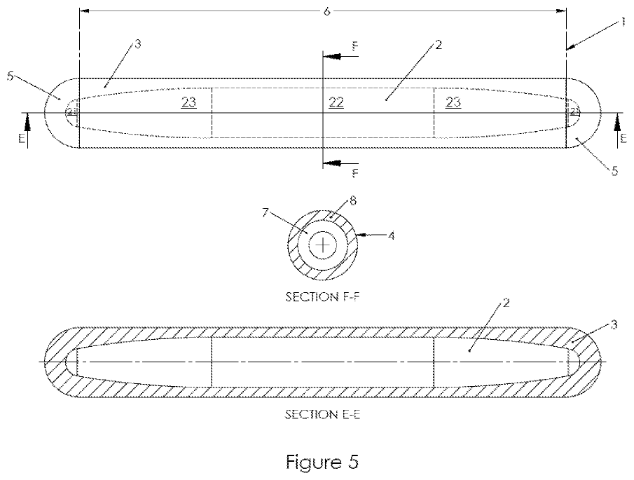

In the examples described herein, the stiffness of the central part 6 of the body at any longitudinal location is related to a ratio of the cross-sectional area 7 of the inner portion 2 to the cross-sectional area 8 of the outer portion 3 at the longitudinal location. The ratio is varied along the length of the member. In the embodiments shown in FIGS. 1, 2, and 4 the ratio is lowest at the longitudinal centre of the body (at section line F-F) and highest at the intersection of the central part 6 and the end 5. This allows the embodiments to have a higher relative stiffness at the longitudinal centre compared to the ends. In the embodiment shown in FIG. 3 the member 1 has a uniform ratio (and constant stiffness) along the length and the embodiment shown in FIG. 5 has the highest ratio (and lowest stiffness) at the longitudinal centre.

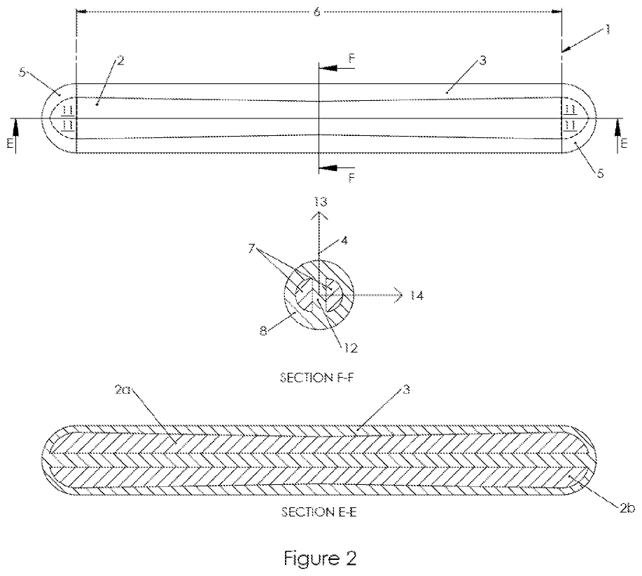

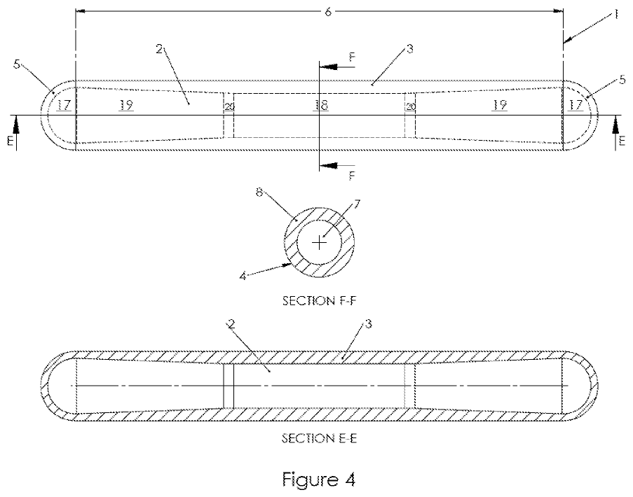

The inner portion 2 may have multiple sections with distinct shapes combined along the length of the member 1 to form the inner portion 2. FIG. 1 shows an embodiment with an inner portion 2 formed by two opposing frustoconical sections 9 with bases oriented towards the ends 5 and apexes oriented toward the centre. The bases have hemispherical sections 10, with a diameter corresponding to the base of the frustoconical section, which extends into the ends. The outer portion 3 extends to the ends 5 and encloses the inner portion 2. FIG. 2 shows an embodiment with two inner portions (2a and 2b). Each portion (2a or 2b) has a semi-circular profile which tapers toward the longitudinal centre and has half hemispherical sections 11 at the ends. The portions (2a and 2b) are separated by a central element 12 which gives the member 2 relatively greater stiffness in one direction 13 as discussed below. FIG. 3 shows an embodiment with an inner portion 2 which is a cylindrical section 15 terminated by two hemispherical ends 16. FIG. 4 shows an embodiment with an inner portion 2 formed by two hemispherical sections 17 at the ends, a central cylindrical section 18, two outer frustoconical sections 19, and two inner frustoconical sections 20 (with a smaller pitch angle) between the outer sections 19 and the central cylindrical section 18. FIG. 5 shows an embodiment with an inner portion formed by two hemispherical sections 21 at the ends, a central cylindrical section 22, and prolate spheroidal frustums 23 between sections 21 and 22. Those skilled in the art will appreciate that the embodiments described above are examples and that many combinations of sections may be used to create an inner portion which satisfies desired requirements.

The longitudinally varying attribute, stiffness in the current example, may be represented as a profile of the attribute in a direction perpendicular to a longitudinal axis of the body along the length of the body. Stiffness, as used herein, refers to the bending stiffness which is related to the second moment of area along a particular direction (13 or 14). The embodiment shown in FIG. 2 has different profiles in at least two directions (13 and 14) perpendicular to the longitudinal axis. The embodiment includes a central beam 12, which increases the stiffness of the member 1 in the direction 13 of the beam 12 when compared to direction 14. This may be useful, for example, where this embodiment is implanted in a patient as a spinal rod with direction 13 parallel to the median plane and direction 14 parallel to the coronal plane of the patient. In this case, the member will provide greater resistance and stiffness to lateral flexion of the spine and less stiffness and resistance to flexion and extension. This is a simplified example of how the profile may be varied in two directions. The member may include a complex internal structure, with complex profiles and bending stiffness in multiple directions as the moment of area is manipulated along the length of the member. This may be desirable, for example, where the member is implanted to correct a localised deformation of a patient's spine and will allow for less stiffness towards the caudal and cephalad ends to allow a less-abrupt and gradual cross-over to normal uninstrumented spinal segments. The moment of inertia and consequent bending stiffness may be increased in multiple directions at the localised deformation and be relaxed and uniform along the remainder of the spine. This will allow the patient to, easily move unaffected portions of the spine, whilst applying effective corrective forces and moments to the localised deformation.

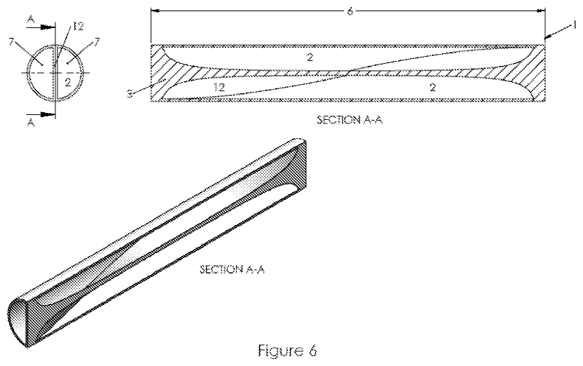

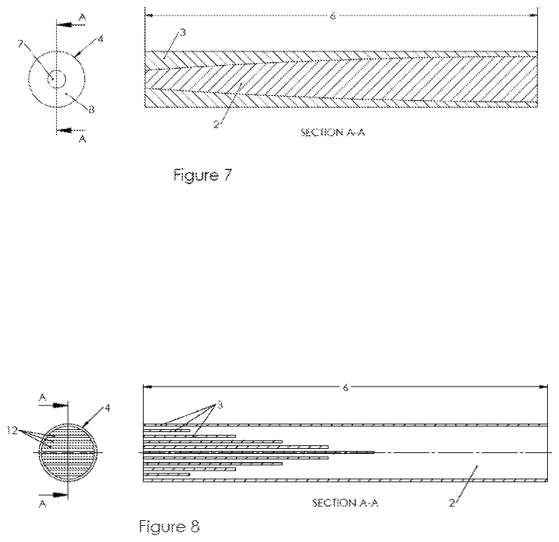

FIG. 6 shows another embodiment which is similar to the embodiment shown in FIG. 2. However, in the embodiment in FIG. 6, the central beam 12 is rotated 180 degrees along the length of the member 1. This creates an internal portion 2 which consists of two twisted semi-circular prisms, wherein the base of the semicircles are parallel, twisted 180 degrees along the longitudinal axis. The effect of this embodiment is that the stiffness is varied along multiple directions along the axis. The stiffness will always be greater in the direction of the central beam 12 and lower in a perpendicular direction. Allowing the stiffness profile in these two directions to be opposites at the center and the ends. FIG. 7 shows another embodiment wherein the internal portion 2 has a substantially frustoconical shape with its base at one end and its apex at the other end of the member 1. This allows the member to have lower stiffness at one end which gradually increases toward the maximum stiffness at the other end. FIG. 8 is another embodiment wherein the outer portion 3 includes a number of parallel beams 12 extending to varying lengths into the inner portion 2. The allows the stiffness profile to be manipulated in two directions by varying the length of the beams 12.

In use, using a patient with scoliosis as an example, the patient's spinal deformation will be measured and quantified by a physician. The physician will determine the required corrective forces and moments to be applied to the deformation. A spinal rod 1 with an inner portion 2 shaped and sized such that the rod 1 as a whole will have the required bending stiffness, density, and other mechanical property profiles which is longitudinally varied to provide the required forces and moments will be created using an additive manufacturing process. The outer portion 3 may be finished and polished to allow for decreased interaction with the patient's tissue.

It is envisaged that the invention will provide a skeletal support member which has varying mechanical properties along the length thereof in at least one direction which may be specifically tailored to allow specific movement and apply corrective forces and moments which are bespoke to the patient.

The invention is not limited to the precise details as described herein. For example, instead of skeletal support members which are spinal rods, the invention may be applied to any stem implants or medullary nails. As a further example, the cross-sectional profile need not be circular, and may be square, hexagonal, or octagonal.

1. A skeletal support member comprising: an elongate body with integrally formed inner and outer portions; at least one mechanical attribute of the inner portion differing from a corresponding mechanical attribute of the outer portion; and the inner portion being shaped and sized such that a corresponding mechanical attribute of the body varies longitudinally; wherein the member is manufactured using an additive manufacturing process.

2. The skeletal support member of claim 1 wherein the mechanical attribute is selected from stiffness, density, strength, ductility, hardness, and surface finish.

3. The skeletal support member of claim 1 wherein the outer portion has a cross-sectional circumferential profile that is longitudinally consistent; or the outer portion has a cross-sectional circumferential profile that is longitudinally varied.

4. The skeletal support member of claim 1 wherein the inner portion is manufactured from a material that is different from a material of the outer portion to vary the mechanical attribute.

5. The skeletal support member of claim 1 wherein the inner portion has a structure which varies the mechanical attribute.

6. The skeletal support member of claim 5 wherein the structure of the inner portion is one or more of a lattice, a grid, a trabecular framework, struts and beams, and sintered.

7. (canceled)

8. (canceled)

9. (canceled)

10. (canceled)

11. The skeletal support member of claim 1 wherein the mechanical attributes of the body at a longitudinal location are related to a ratio of a cross-sectional area of the inner portion to cross-sectional area of the outer portion at the location.

12. The skeletal support member of claim 11 wherein the ratio is varied along the length of the member; or
the ratio is lowest at a central part of the body and highest at the ends of the body; or
the ratio is varied along the length of the body to impart specific deforming or deformity opposing forces; or
the ratio is highest at a central part of the body and lowest at the ends of the body.

13. (canceled)

14. (canceled)

15. The skeletal support member of claim 1 wherein the inner portion includes multiple sections with distinct shapes combined along the length of the member to form the inner portion.

16. The skeletal support member of claim 1 wherein the inner portion is formed by combining two opposing tapered sections with bases oriented towards the ends of the body and apexes oriented toward the center of the body.

17. The skeletal support member of claim 16 wherein the shape of the tapered sections are selected from pyramidal, conical, frustopyramidal, and frustoconical shapes.

18. The skeletal support member of claim 1 wherein the outer portion extends to the ends of the body and enclose the inner portion.

19. The skeletal support member of claim 1 wherein the outer portion has a lower surface roughness than a surface roughness of the inner portion.

20. The skeletal support member of claim 1 wherein the longitudinally varying attribute is represented as a profile of the attribute in a direction perpendicular to a longitudinal axis of the body along the length of the body.

21. The skeletal support member of claim 1 wherein the member is made of titanium.

22. The skeletal support member of claim 1 wherein the additive manufacturing process is laser metal deposition.

23. The skeletal support member of claim 1 wherein the body is cylindrical with rounded ends and a solid outer portion.

24. The skeletal support member of claim 1 wherein the member is 3D printed such that the outer portion transitions to the inner portion gradually.

25. The skeletal support member of claim 1 wherein one or more of the inner portion has a relatively lower density and stiffness than the outer portion; and the body has a relatively higher stiffness at the center and relatively lower stiffness at the ends.

26. (canceled)

27. The skeletal support member of claim 1 wherein the member is one or more of a spinal rod, an intramedullary nail, or a prosthetic stem.

28. (canceled)

29. (canceled)
